(19) United States
(12) Patent Application Publication
(10) Pub. No.: US 2025/0319455 A1
(43) Pub. Date: Oct. 16, 2025

(54) Method

(71) Applicant: Mexichem Fluor S.A. de C.V., San Luis Potosi (MX)

(72) Inventors: Andrew Sharratt, Runcorn (GB); Claire Rees, Runcorn (GB)

(21) Appl. No.: 19/251,054

(22) Filed: Jun. 26, 2025

Related U.S. Application Data
(63) Continuation of application No. 17/418,926, filed on Jun. 28, 2021, now Pat. No. 12,357,968.

(30) Foreign Application Priority Data
Jan. 17, 2019 (GB) 1900647.7

(51) Int. Cl. B01J 23/26; B01J 23/06; B01J 35/40; B01J 35/50; B01J 35/63; B01J 35/64; B01J 37/06; B01J 37/14; C07C 17/07

(52) U.S. Cl. CPC B01J 23/26; B01J 23/06; B01J 35/40; B01J 35/633; B01J 35/653; B01J 37/06; B01J 37/14; C07C 17/07; B01J 35/50; B01J 2235/15

(57) ABSTRACT

A method for activating a catalyst comprises the steps of: optionally drying the catalyst at a temperature of from 100° C. to 400° C.; treating the catalyst with a composition comprising HF at a temperature of from 500° C. to about 700° C.; and optionally treating the catalyst with a composition comprising an oxidant and optionally HF at a temperature of from about 100° C. to about 500° C.

### RELATED APPLICATIONS

The present application is a continuation of U.S. application Ser. No. 17/418,926, filed Jan. 16, 2020, which is the U.S. national phase of PCT international Application No. PCT/GB2020/050087, filed Jan. 16, 2020, which are herein incorporated by reference in its entirety.

### TECHNICAL FIELD

The present invention relates to a method for activating a catalyst and to a process that uses said catalyst.

### BACKGROUND

The listing or discussion of a prior published document in this specification should not necessarily be taken as an acknowledgement that the document is part of the state of the art or is common general knowledge.

Halocarbon-based compounds, particularly fluorocarbon-based compounds are currently used in a large number of commercial and industrial applications, such as propellants, blowing agents and heat transfer fluids. The interest in and use of fluorine-based compounds, particularly (hydro)fluoroolefins, as heat transfer fluids has increased as new refrigerants are sought.

Fluorination and/or hydrofluorination steps are also common in the manufacturing processes of (hydro)fluoroalkenes. Such processes may be performed by contacting HF with one or more (hydro)haloalkenes or (hydro)haloalkanes, preferably in the presence of a catalyst.

Fluorination and/or hydrofluorination steps involve reacting a starting material with a fluorinating agent (e.g. hydrogen fluoride) to introduce one or more fluorine atoms to the starting material. Such processes comprise addition of one or more fluorine atoms to the starting material and/or substitution of one or more atoms of the starting material with one or more fluorine atoms.

Typically, fluorination and/or hydrofluorination steps are performed industrially under catalysis. Catalysts suitable for use in such processes are often compounds of transition metals, for instance transition metal oxides and/or halides. Preferred examples of such catalysts include those based on chromia, frequently containing another metal, such as zinc.

Prior to the fluorination and/or hydrofluorination step, the catalyst is usually subjected to an activation treatment to achieve the desired catalytic performance. Normally, this involves treating the catalyst with hydrogen fluoride, at an elevated temperature. Frequently, the activation treatments are preceded by other steps, such as drying or heating the catalyst in inert atmosphere, in other words “calcination”.

WO2010/026382 discloses such methods for treating a catalyst for use prior to engaging a catalyst in fluorination of 1-chloro-2,2,2-trifluoroethane to 1,1,1,2-tetrafluoroethane (R-134a). In EP 06726692, a catalyst is treated prior to contacting the catalyst with hydrogen fluoride and perchloroethylene to yield pentafluoroethane.

However, it has been found that these activation treatments are insufficient to successfully activate catalysts for the processes of fluorinating and/or hydrofluorinating halogenated hydrocarbons with longer carbon chains, particularly C₃₋₇(hydro)haloalkenes. The catalysts have been found to suffer from slow and complex activation behaviour, lack of stability, as well as mass transport limitations. All these limitations result in poor catalytic performance. These problems have been observed in processes of commercial interest, particularly in conversion of 2-chloro-3,3,3-trifluoropropene (1233xf) to 2,3,3,3-tetrafluoropropene (1234yf). Therefore, there exists a need for developing improved methods for activating fluorination catalysts.

Further, catalytic reactions involving halocarbons have a number of problems in use, one of which is that industrial scale processes subject the catalysts to extreme temperatures and pressures, numerous regenerations and corrosive reagents. The skilled person will know that over the lifetime of an industrial catalyst the activity is steadily reduced, and the catalyst must eventually be replaced in an expensive procedure.

There is therefore a need for catalysts with improved stability and comparable or improved activity and selectivity relative to existing catalysts.

### DETAILED DESCRIPTION

According to a first aspect of the invention there is provided a method for activating a catalyst comprising the steps of:
a) optionally drying the catalyst at a temperature of from 100° C. to 400° C.;
b) treating the catalyst with a composition comprising HF at a temperature of from 100° C. to about 500° C.;
c) treating the catalyst with a composition comprising an oxidant and optionally HF at a temperature of from about 100° C. to about 500° C.

The oxidant is preferably selected from air, oxygen (O₂), chlorine (Cl₂), chlorine monofluoride (ClF), nitrogen trifluoride (NF₃) and combinations thereof.

The molar ratio of HF to oxidant is preferably from 1:20 to 20:1, more preferably from 15:1 to 1:3, and most preferably from 11:1 to 1:1.

Step (b) and/or (c) is preferably carried out at a pressure of from 0.1 bara to 20 bara, more preferably from 3 bara to 10 bara.

Step (b) and/or (c) is preferably carried out over an extended period. The period will be recognised by the skilled person as being scale dependent. At laboratory scale a duration of between about 5 hours and about 65 hours is preferred, more preferably between about 8 hours and about 55 hours. In a commercial operating plant this timing may require extension. In a commercial operating plant, a duration of between about 24 hours and about 168 hours is preferred. The length of time employed for step (b) and for step (c) may be the same or different.

Step (b) and/or (c) is preferably carried out at a temperature of from about 200° C. to about 500° C., more preferably from about 250° C. to about 475° C., more preferably from about 300° C. to 460° C., such as from about 310° C. to about 450° C. The temperature of step (b) and step (c) may be the same or different.

According to a second aspect of the invention there is provided a method for activating a catalyst comprising the steps of:
a) optionally drying the catalyst at a temperature of from 100° C. to 400° C.;
b) treating the catalyst with a composition comprising HF at a temperature of from 500° C. to about 700° C.

In the method of the second aspect of the invention, step (b) is preferably carried out at a pressure of from 0.1 bara to 20 bara, more preferably from 3 bara to 10 bara.

In the method of the second aspect of the invention, step (b) is preferably carried out over an extended period. The period will be recognised by the skilled person as being scale dependent. At laboratory scale a duration of between about 5 hours and about 65 hours is preferred, more preferably between about 8 hours and about 55 hours. In a commercial operating plant this timing may require extension. In a commercial operating plant a duration of between about 24 hours and about 168 hours is preferred. It will be appreciated that the length of time employed for step (b) and for step (c) may be the same or different.

In the method of the second aspect of the invention, step (b) is preferably carried out at a temperature of from about 500° C. to about 600° C., most preferably about 520° C.

Preferably the catalyst comprises chromia and at least one additional metal or compound thereof, wherein the at least one additional metal is selected from Li, Na, K, Ca, Mg, Cs, Sc, Al, Y, Ti, Zr, Hf, V, Nb, Ta, Cr, Mo, W, Mn, Re, Fe, Ru, Co, Rh, Ir, Ni, Pd, In, Pt, Cu, Ag, Au, Zn, La, Ce and mixtures thereof.

In a further aspect, there is provided a method for producing 2,3,3,3-tetafluoropropene (1234yf) from a precursor in the presence of a catalyst activated in accordance with the first aspect of the invention. This additional metal or compound thereof can also be referred to as a promoter. Preferably, the at least one additional metal is selected from Li, Na, K, Ca, Mg, Cs, Cr, Zr, Nb, Pd, Ta, Zn, V, Mo, Ni, Co, In, Fe, Cu and mixtures thereof, even more preferably the additional metal is zinc. The skilled person would appreciate that in catalysis in general, catalytic activity is understood to be proportional to the available surface area of the catalyst. It is to be expected that increasing the opportunity for the reagents to interact with the surface of the catalyst will improve the rate of conversion. However, in contrast to established teaching, the present inventors have surprisingly found that increasing the pore volume and average pore diameter, which may inherently reduce a catalyst's surface area, increases both the stability and the activity of the catalyst.

Without wishing to be bound by theory, it is believed that this is a result of the increased mass transfer through the catalyst and that this effect is more pronounced for C₃ compounds than C₂ compounds. Also, without wishing to be bound by theory, it is believed that the wider pore diameters of the present invention allow the catalyst in use to assume more quickly an effective pore structure for producing (hydro)haloalkenes such as hydrofluoropropenes.

The pore structure of solid porous materials can be determined by several methods, one of the most commonly used is the adsorption and desorption of N₂, based on the BET theory (Brunauer, Emmett and Teller) of the adsorption of multilayers of condensed gases onto solid surfaces, and the evaporation (desorption) of the adsorbed gas during desorption. Nitrogen is a common adsorbate for probing the micro and mesoporous regions. From the adsorption and desorption isotherms, the following can be calculated: BET surface area from the adsorption of a monolayer of N₂, total pore volume taken from the amount of nitrogen adsorbed at P/Pº=0.99 and average pore diameters can be determined using different calculations either based on the BET theory or that of BJH (Barrett, Joyner and Halenda), either from the adsorption or desorption data. Preferably, the total pore volume of the catalyst is equal to or greater than 0.35 cm³/g or 0.4 cm³/g, such as 0.45 cm³/g, 0.5 cm³/g, 0.55 cm³/g or even 0.6 cm³/g when measured by N₂ adsorption porosimetry.

Preferably, the average pore width of the catalyst is greater than or equal to 100 Å, e.g. greater than or equal to 110 Å or greater than or equal to 120 Å when measured by N₂ BET adsorption porosimetry.

Preferably, the average pore width of the catalyst is greater than or equal to 130 Å, e.g. greater than or equal to 140 Å, greater than or equal to 150 Å or greater than or equal to 170 Å when measured by N₂ BJH adsorption porosimetry.

Preferably, the average pore width of the catalyst is greater than or equal to 90 Å, e.g. greater than or equal to 100 Å, greater than or equal to 110 Å or greater than or equal to 120 Å when measured by N₂ BJH desorption porosimetry.

It is appreciated that other methods of porosimetry measurements are known to the skilled person.

The catalysts preferably have a surface area from 5 to 250 m²/g following activation. Typically, the catalysts have a surface area of 40 to 50 m²/g following activation.

Preferably, the catalyst is provided in the form of a pellet or pellets comprising a plurality of catalyst particles. Such catalyst particles may be pressed together, for example under load, to form the pellets. The pellets may comprise one or more further materials. For example, the pellets may include graphite, preferably in an amount of from about 0.5 wt % to about 10 wt %, e.g. from about 1 wt % to about 5 wt %. Preferably, the pellets have a longest dimension from about 1 mm to about 100 mm. In some embodiments, the pellets may have a longest dimension of about 1 mm to about 10 mm, for example from about 3 mm to about 5 mm.

Preferably, the catalyst comprises at least 80 wt % (for example at least 85 wt %, at least 90 wt %, at least 92 wt %, at least 93 wt %, at least 94 wt %, at least 95 wt % or at least 96 wt %) chromia. Advantageously, the catalyst may be a zinc/chromia catalyst. By the term “zinc/chromia catalyst” we mean that the metal oxide catalyst comprises chromium or a compound of chromium and zinc or a compound of zinc.

The total amount of the zinc or a compound of zinc present in the zinc/chromia catalysts of the invention is typically from about 0.01% to about 25%, preferably 0.1% to about 25%, conveniently 0.01% to 6% of the catalyst; and in some embodiments preferably 0.5% by weight to about 25% by weight of the catalyst, preferably from about 1 to 10% by weight of the catalyst, more preferably from about 2 to 8% by weight of the catalyst, for example about 3 to 6% by weight of the catalyst.

In further preferred embodiments, the additional metal compound may comprise indium (e.g. in the form In₂O₃) and/or zirconium (e.g. in the form ZrO₂).

Additional metals or compounds thereof are typically present from about 0.01% to about 25%, preferably 0.1% to about 25%, conveniently 0.01% to 6% by weight of the catalyst; and in some embodiments preferably 0.5% by weight to about 25% by weight of the catalyst, preferably from about 1 to 10% by weight of the catalyst, more preferably from about 2 to 8% by weight of the catalyst, for example about 4 to 6% by weight of the catalyst.

In other embodiments, the catalyst may be an alumina catalyst with one or more promoters selected from platinum, iron, chromium and zinc. The total amount of promoter is typically from about 0.1 to about 60% by weight of the catalyst, preferably from about 0.5 to about 50% by weight of the catalyst, such as 0.5% by weight to about 25% by weight of the catalyst, or from about 1 to 10% by weight of the catalyst. In such embodiments it is preferred that the catalyst comprises at least 80 wt % (for example at least 85 wt %, at least 90 wt %, at least 92 wt %, at least 93 wt %, at least 94 wt %, at least 95 wt % or at least 96 wt %) chromia. In some embodiments, the catalyst may be in fluorinated form. For example, the catalyst may have been fluorinated by treatment with HF at elevated temperature.

Many of these preferred features may be combined into a preferred embodiment. Thus, according to a third aspect of the invention there is provided a method for activating a catalyst, wherein the catalyst comprises a chromia catalyst, with zinc oxide present in the catalyst at a level of from 1% wt to 10% wt, wherein the catalyst has a total pore volume equal to or greater than 0.3 cm³/g and a mean pore diameter greater than or equal to 100 Å, wherein the pore volume is measured using N₂ adsorption porosimetry and the mean pore diameter is measured using N₂ BET adsorption porosimetry; comprising the steps of:
a) optionally drying the catalyst at a temperature of from 100° C. to 400° C.;
b) treating the catalyst with a composition comprising HF at a temperature of from 100° C. to about 500° C.;
c) treating the catalyst with a composition comprising an oxidant and optionally HF at a temperature of from about 100° C. to about 500° C.

Preferred features of the first and second aspects of the invention shall; be taken to apply mutatis mutandis to the third aspect of the invention.

Preferably the zinc oxide/chromia catalyst used in the present invention may be amorphous. By this we mean that the catalyst does not demonstrate any substantial crystalline characteristics when analysed by, for example, X-ray diffraction.

Alternatively, the zinc oxide/chromia catalyst used in the present invention may be partially crystalline. By this we mean that from 0.1 to 50% by weight of the catalyst is in the form of one or more crystalline compounds of chromium and/or one or more crystalline compounds of zinc. If a partially crystalline catalyst is used, it preferably contains from 0.2 to 25% by weight, more preferably from 0.3 to 10% by weight, still more preferably from 0.4 to 5% by weight of the catalyst in the form of one or more crystalline compounds of chromium and/or one or more crystalline compounds of zinc.

It is appreciated that during use in a reaction the degree of crystallinity may change. Thus it is possible that a catalyst having a degree of crystallinity as defined above before use in a reaction and will have a degree of crystallinity outside these ranges during or after use in a reaction.

The catalysts preferably have a surface area from 5 to 250 m²/g following activation. Typically, the catalysts have a surface area of 40 to 50 m²/g following activation.

The present invention also provides a method of preparing a catalyst, said method comprising the steps of:
a) preparing a metal salt solution and a hydroxide solution;
b) combining the solutions at a pH of greater than 7.5 in order to precipitate the metal hydroxide(s);
c) drying the precipitated metal hydroxides;
d) calcining the metal hydroxide(s) to form the metal oxide(s).

Preferably, the metal salt comprises a nitrate salt such as a hydroxide nitrate salt. In preferred embodiments, the metal salt comprises chromium, and may comprise a chromium nitrate salt such as Cr(OH)(NO₃)₂. The hydroxide solution may comprise ammonium hydroxide (NH₄OH). Advantageously, step b) is carried out at a pH of greater than 8. Preferably, step b) is carried out at a pH of greater than or equal to 8.1, 8.2, 8.3; 8.4 or 8.5.

In a further aspect of the invention, there is provided a process for fluorinating a C₂₋₃ hydrohalocarbon species, comprising contacting the species with a catalyst according to the invention. This is typically carried out in the presence of HF. For the avoidance of doubt, the term C₂₋₃ hydrohalocarbon includes saturated or unsaturated compounds with a two or three carbon chain and containing one or more atoms of hydrogen and a halogen (F, CI, Br, I). In preferred embodiments, the hydrohalocarbon species comprises a C₃ hydrohalocarbon species.

An example of such a process comprises contacting trichloroethylene with the catalyst in the presence of HF to produce 1,1,1,2-tetrafluoroethane (134a), the conversion of 1,1,1,2,3-pentachloropropane (240 db) to 2-chloro-3,3,3-trifluoropropene (1233xf), the conversion of 1233xf to 2,3,3,3-tetrafluoropropene (1234yf) and/or 1,1,1,2,2-pentfluoropropane (245cb), the conversion of 1,1,1,3-tetrachloropropane (250fb) to 3,3,3-trifluoropropene (1243zf), or the conversion of 2,3-dichloro-1,1,1-trifluoropropane (243 db) to 1233xf and/or 1234yf.

In another aspect of the invention, there is provided a process for dehydrohalogenating a C₂₋₃ hydrohalocarbon species (preferably a C₃ hydrohalocarbon species), comprising contacting the species with a catalyst, such as contacting a hydro(halo)fluoropropane with the catalyst to produce a fluoropropene, preferably a tetrafluoropropene (1234) such as 1234ze ((E) or (Z)) or 1234yf. Advantageously, this may include the conversion of 245cb and/or 1,1,1,2,3-pentafluoropropane (245eb) to 2,3,3,3-tetrafluoropropene (1234yf) and/or 1,3,3,3-tetrafluoropropene (1234ze), the conversion of 1,1,1,3,3-pentafluoropropane (245fa) to 1234ze or the conversion of 1-chloro-1,3,3,3-tetrafluoropropane to 1-chloro-3,3,3-trifluoropropene (1233zd) or 1234ze. In a further aspect of the invention, there is provided a process for eliminating HF or from a saturated C₂₋₃ hydrohalocarbon species (preferably a C₃ hydrohalocarbon species), comprising contacting the species with a catalyst

In another aspect of the invention, there is provided a process for adding HF to an unsaturated C₂₋₃ hydrohalocarbon species (preferably a C₃ hydrohalocarbon species), comprising contacting the species with a catalyst.

The processes may be conducted in the liquid or the vapour phase but are preferably conducted in the vapour phase. The process may be carried out at atmospheric, sub-or super atmospheric pressure, typically at from 0 to about 30 bara, preferably from about 1 to about 20 bara, such as 15 bara.

Typically, the vapour phase process is carried out a temperature of from about 100° C. to about 500° C. (e.g. from about 150° C. to about 500° C. or about 100 to about 450° C.). Preferably, the process is conducted at a temperature of from about 150° C. to about 450° C., such as from about 150° C. to about 400° C., e.g. from about 175° C. to about 300° C. Lower temperatures may also be used in the conversion of 250fb to 1243zf, such as from about 150° C. to about 350° C., e.g. from about 150° C. to about 300° C. or from about 50° C. to about 250° C.

The processes typically employ a molar ratio of HF: organics of from about 1:1 to about 100:1, such as from about 3:1 to about 50:1, e.g. from about 4:1 to about 30:1 or about 5:1 or 6:1 to about 20:1 or 30:1. The reaction time for the process generally is from about 1 second to about 100 hours, preferably from about 10 seconds to about 50 hours, such as from about 1 minute to about 10 or 20 hours. In a continuous process, typical contact times of the catalyst with the reagents are from about 1 to about 1000 seconds, such from about 1 to about 500 seconds or about 1 to about 300 seconds or about 1 to about 50, 100 or 200 seconds.

### EXAMPLES

### Catalyst Activation Examples

3 ml of chromia catalyst particles (size range from 0.5 mm to 1.0 mm) containing 3 wt % ZnO was dried by heating at 250° C. for 4 hours under a nitrogen atmosphere (60 ml/min) at 1 Bara.

The catalyst was subjected to a first activation step at 380° C. for 16 hours under a HF atmosphere (30 ml/min). The catalyst was subjected to a second activation step under the conditions as outlined below.

| | Second Activation Step | | |
|---|---|---|---|
| Example | Temperature | Duration | Activants |
| 1 | 380° C. | 16 hours | Air and HF (25 ml/min each) |
| 2 | 380° C. | 40 hours | Air and HF (25 ml/min each) |
| 3* | 380° C. | 16 hours | Air and HF (25 ml/min each) |
| 4* | 380° C. | 40 hours | Air and HF (25 ml/min each) |

*the chromia contained 6.5 wt % ZnO.

| Comparative | Second Activation Step | | | |
|---|---|---|---|---|
| Example | Temperature | Duration | Activants | |
| A | 380° C. | 16 hours | Nitrogen and HF (25 | ml/min each) |
| B | 380° C. | 16 hours | Nitrogen and Air (25 | ml/min each) |
| C | 380° C. | 16 hours | Nitrogen (50 | ml/min) |
| D | 380° C. | 40 hours | Nitrogen and HF (25 | ml/min each) |
| E | 380° C. | 40 hours | Nitrogen and Air (25 | ml/min each) |
| F | 380° C. | 40 hours | Nitrogen (50 | ml/min) |
| G* | 380° C. | 16 hours | Nitrogen and HF (25 | ml/min each) |
| H* | 380° C. | 16 hours | Nitrogen and Air (25 | ml/min each) |
| I* | 380° C. | 16 hours | Nitrogen (50 | ml/min) |
| J* | 380° C. | 40 hours | Nitrogen and HF (25 | ml/min each) |
| K* | 380° C. | 40 hours | Nitrogen and Air (25 | ml/min each) |
| L* | 380° C. | 40 hours | Nitrogen (50 | ml/min) |

*the chromia contained 6.5 wt % ZnO.

### Catalytic Examples

The activated catalyst was contacted with 2-chloro-3,3,3-trifluoropropene (1233xf) (1 ml/min) and hydrogen fluoride (25 ml/min) in a reactor. The cycle time was between about 21 hours and about 28 hours. The pressure was 1 bara.

The data generated was in the form of reactor off-gas (ROG) compositions for the feed (1233xf) and main reaction products 2,3,3,3-tetrafluoropropene (1234yf) and 1,1,1,2,2-pentafluoropropane (245cb).

### Results

| | Cycle time | 1233xf | 1234yf | 245cb | |
|---|---|---|---|---|---|
| Catalyst | (hrs) | (mol %) | (mol %) | (mol %) | k′_{xf} |
| 1 | 0.5 | 23.63 | 59.29 | 17.08 | 1.12 |
| | 2.06 | 28.71 | 56.03 | 15.26 | 0.94 |
| | 3.38 | 30.96 | 54.29 | 14.76 | 0.94 |
| | 4.7 | 31.10 | 54.30 | 14.60 | 0.93 |
| | 21.1 | 36.07 | 50.37 | 13.56 | 0.83 |
| 2 | 0.83 | 29.14 | 55.15 | 15.71 | 1.01 |
| | 3.12 | 31.35 | 53.63 | 15.01 | 0.95 |
| | 4.86 | 31.64 | 53.50 | 14.85 | 0.99 |
| | 21.38 | 34.74 | 51.14 | 14.11 | 0.81 |
| | 27 | 36.24 | 49.98 | 13.77 | 0.76 |
| 3* | 1.13 | 28.74 | 53.59 | 17.67 | 1.03 |
| | 3.65 | 31.79 | 51.38 | 16.82 | 0.97 |
| | 5.3 | 32.88 | 50.62 | 16.50 | 0.90 |
| | 22.13 | 47.94 | 39.63 | 12.43 | 0.47 |
| | 24.43 | 50.55 | 37.71 | 11.74 | 0.43 |
| 4* | 0.76 | 29.13 | 54.26 | 16.61 | 0.94 |
| | 2.86 | 33.55 | 51.43 | 15.01 | 0.81 |
| | 23.65 | 43.10 | 44.36 | 12.54 | 0.60 |

| | Treatment | Cycle | 1233xf | 1234yf | 245cb | |
|---|---|---|---|---|---|---|
| Catalyst | time (hrs) | time (hrs) | (mol %) | (mol %) | (mol %) | k′_{xf} |
| A | 16 | 0.82 | 31.83 | 52.99 | 15.18 | 0.82 |
| | 16 | 2.23 | 34.14 | 51.25 | 14.60 | 0.72 |
| | 16 | 3.56 | 36.42 | 49.65 | 13.93 | 0.71 |
| | 16 | 4.88 | 37.60 | 48.72 | 13.68 | 0.69 |
| | 16 | 21.35 | 44.83 | 43.05 | 12.11 | 0.54 |
| B | 16 | 1.06 | 27.82 | 55.37 | 16.81 | 0.89 |
| | 16 | 2.48 | 33.60 | 50.98 | 15.43 | 0.73 |
| | 16 | 3.82 | 39.54 | 46.55 | 13.91 | 0.60 |
| | 16 | 5.12 | 45.62 | 41.95 | 12.44 | 0.48 |
| | 16 | 21.52 | 78.37 | 16.83 | 4.78 | 0.13 |
| C | 16 | 1.25 | 57.58 | 32.09 | 10.33 | 0.30 |
| | 16 | 2.65 | 68.17 | 24.40 | 7.42 | 0.20 |
| | 16 | 3.98 | 72.70 | 21.06 | 6.25 | 0.16 |
| | 16 | 5.32 | 74.48 | 19.71 | 5.81 | 0.16 |
| | 16 | 21.75 | 77.31 | 17.62 | 5.07 | 0.14 |
| D | 40 | 0.5 | 36.04 | 49.97 | 13.99 | 0.71 |
| | 40 | 2.92 | 37.61 | 48.63 | 13.75 | 0.76 |
| | 40 | 4.7 | 38.02 | 48.42 | 13.56 | 0.73 |
| | 40 | 21.1 | 41.74 | 45.50 | 12.76 | 0.62 |
| | 40 | 26.78 | 42.29 | 45.11 | 12.59 | 0.62 |
| E | 40 | 1.38 | 34.67 | 49.35 | 15.98 | 0.77 |
| | 40 | 3.48 | 47.01 | 40.08 | 12.89 | 0.48 |
| | 40 | 5.32 | 63.56 | 27.69 | 8.75 | 0.25 |
| | 40 | 21.78 | 79.24 | 16.12 | 4.63 | 0.12 |
| | 40 | 27.45 | 79.36 | 16.05 | 4.59 | 0.12 |
| F | 40 | 1.22 | 52.51 | 36.37 | 11.12 | 0.38 |
| | 40 | 3.26 | 62.89 | 28.61 | 8.49 | 0.25 |
| | 40 | 5.12 | 66.87 | 25.64 | 7.49 | 0.22 |
| | 40 | 21.56 | 73.37 | 20.69 | 5.94 | 0.17 |
| | 40 | 27.26 | 73.61 | 20.47 | 5.92 | 0.17 |
| G | 16 | 0.92 | 27.64 | 54.83 | 17.51 | 1.04 |
| | 16 | 3.47 | 31.48 | 52.23 | 16.29 | 0.96 |
| | 16 | 5.02 | 32.32 | 51.65 | 16.03 | 0.89 |
| | 16 | 21.92 | 46.46 | 41.07 | 12.47 | 0.49 |
| | 16 | 24.23 | 48.78 | 39.37 | 11.84 | 0.44 |
| H | 16 | 0.6 | 30.80 | 52.49 | 16.71 | 0.87 |
| | 16 | 3.22 | 43.99 | 43.03 | 12.97 | 0.54 |
| | 16 | 4.83 | 55.67 | 34.23 | 10.09 | 0.35 |
| | 16 | 21.73 | 80.14 | 15.57 | 4.29 | 0.12 |
| | 16 | 24.03 | 80.04 | 15.65 | 4.30 | 0.12 |
| I | 16 | 0.5 | 30.20 | 53.80 | 15.99 | 0.91 |
| | 16 | 3.05 | 36.33 | 49.99 | 13.67 | 0.79 |
| | 16 | 4.62 | 41.97 | 45.55 | 12.48 | 0.62 |
| | 16 | 21.55 | 72.69 | 21.51 | 5.79 | 0.19 |
| | 16 | 23.88 | 72.60 | 21.57 | 5.82 | 0.18 |
| J | 40 | 1 | 27.66 | 56.52 | 15.81 | 0.98 |
| | 40 | 3.15 | 31.29 | 53.94 | 14.76 | 0.85 |
| | 40 | 23.85 | 39.72 | 47.36 | 12.91 | 0.68 |
| K | 40 | 0.62 | 34.20 | 51.44 | 14.36 | 0.76 |
| | 40 | 2.65 | 48.32 | 40.50 | 11.18 | 0.46 |
| | 40 | 23.45 | 81.76 | 14.43 | 3.81 | 0.12 |
| L | 40 | 1.2 | 32.34 | 52.62 | 15.03 | 0.89 |
| | 40 | 3.35 | 39.99 | 46.74 | 13.26 | 0.67 |
| | 40 | 24.16 | 74.73 | 19.82 | 5.45 | 0.16 |

For each data point the equilibrium position of the reaction was calculated and then the instantaneous net rate constant for 1233xf conversion, k′_{xf}, was calculated

In this way for each experiment it was possible to quantify catalyst effectiveness vs time and so derive useful catalyst performance characteristics relating to activity and stability.

For each experiment a plot of k′_{xf} vs time was produced and fitted using the equation:

$${k}^{′} \times f = b + ( a - b ) {exp}^{- {k}^{″}} t$$

Where:
   a=initial activity
   b=final activity
   k″=catalyst activity decay rate
and where the initial rate of catalyst activity decay at t=0 hrs=

$$- \frac{{k}^{″} ( a - b )}{{exp}^{- {k}^{″} t}}$$

This is exemplified in the table below

| | Initial | Final | Rate constant | k′_{xf} | Initial rate |
|---|---|---|---|---|---|
| | Activity | Activity | decay rate | (24 hr) | k′_{xf} |
| Catalyst | (s⁻¹) | (s⁻¹) | (hr⁻¹) | (s⁻¹) | decay |
| 1 | 1.1608 | 0.8447 | 0.3949 | 0.8447 | −0.1248 |
| 2 | 1.0115 | 0 | 0.0105 | 0.7862 | −0.0106 |
| 3* | 1.0941 | 0 | 0.0378 | 0.4416 | −0.0414 |
| 4* | 1.0122 | 0.6018 | 0.2356 | 0.6032 | −0.0967 |
| A | 0.8377 | 0.5329 | 0.1523 | 0.5408 | −0.0464 |
| B | 1.0617 | 0.1095 | 0.1775 | 0.1229 | −0.1690 |
| C | 0.5585 | 0.1432 | 0.7607 | 0.1432 | −0.3159 |
| D | 0.7468 | 0 | 0.0074 | 0.6253 | −0.0055 |
| E | 1.1896 | 0.1195 | 0.3471 | 0.1198 | −0.3714 |
| F | 0.5057 | 0.1693 | 0.3983 | 0.1693 | −0.1340 |
| G | 1.0769 | 0 | 0.0356 | 0.4583 | −0.0383 |
| H | 0.9966 | 0.1174 | 0.2508 | 0.1195 | −0.2205 |
| I | 0.9646 | 0.0713 | 0.0922 | 0.1690 | −0.0824 |
| J | 1.0678 | 0.6747 | 0.2538 | 0.6756 | −0.0998 |
| K | 0.8959 | 0.1143 | 0.3037 | 0.1148 | −0.2374 |
| L | 1.044 | 0.146 | 0.1619 | 0.1644 | −0.1454 |

The catalytic example was repeated under the same conditions as above with the exception that the pressure was increased to 6 bara. The kinetic data obtained is shown in the table below.

| | | | Rate |
|---|---|---|---|
| | Initial | Final | constant |
| | activity | activity | decay rate |
| Catalyst | (s⁻¹) | (s⁻¹) | (hr⁻¹) |
| C | 0.0143 | 0.0000 | 0.0131 |
| 1 | 0.3696 | 0.0000 | 0.0233 |

The catalysts activated according to the method of the invention not only show high initial activity but also show a lower decrease in said activity when compared to catalysts activated in a comparative method.

### Pore Size Measurements

Pore sizes of the catalyst were measured according to the BET Ads (4V/A) method. The results are shown in the table below.

| | Average pore diameter |
|---|---|
| | BET Ads (4V/A) |
| Catalyst | (Å) |
| 1 | 142.5 |
| 2 | 151.6 |
| 3* | 146.6 |
| 4* | 146.7 |
| A | 130.4 |
| B | 175.2 |
| C | 133.6 |
| D | 139.6 |
| E | 184.4 |
| F | 142.7 |
| G | 150.3 |
| H | 170.5 |
| I | 144.6 |
| J | 150.5 |
| K | 170.7 |
| L | 144.8 |

Thus, it has been demonstrated how catalyst activation treatments according to the teaching of this patent result in working catalysts with increased porosity and enhanced activity and stability.

1. (canceled)

2. (canceled)

3. (canceled)

4. (canceled)

5. The method according to claim 12, wherein the catalyst is a chromia catalyst.

6. The method according to claim 5, wherein the catalyst is a chromia catalyst comprising zinc or a compound thereof, such as zinc oxide.

7. The method according to claim 6, wherein zinc oxide is present in the catalyst at a level of from 1% wt to 10% wt, more preferably from 2% wt to 8% wt, more preferably from 3% wt to 7% wt based on the total weight of the catalyst.

8. (canceled)

9. (canceled)

10. (canceled)

11. (canceled)

12. A method for activating a catalyst comprising:
(a) optionally drying the catalyst at a temperature of from 100° C. to 400° C.;
(b) treating the catalyst with a composition comprising HF at a temperature of from 500° C. to about 700° C.

13. The method according to claim 12, wherein step (b) is carried out at a pressure of from 0.1 bara to 20 bara, preferably from 3 bara to 10 bara.

14. The method according to claim 12, wherein step (b) is carried out over an extended period of time.

15. The method according to claim 12, wherein step (b) is carried out at a temperature of from about 500° C. to about 600° C.

16. The method according to claim 12, wherein the catalyst comprises a chromia catalyst, with zinc oxide present in the catalyst at a level of from 1% wt to 10% wt, wherein the catalyst has a total pore volume equal to or greater than 0.3 cm³/g and a mean pore diameter greater than or equal to 100 A, wherein the pore volume is measured using N₂ adsorption porosimetry and the mean pore diameter is measured using N₂ BET adsorption porosimetry.

17. The method according to claim 16, wherein the catalyst is prepared by
a) preparing a metal salt solution and a hydroxide solution;
b) combining the solutions at a pH of greater than 7.5 in order to precipitate the metal hydroxide(s);
c) drying the precipitated metal hydroxides;
d) calcining the metal hydroxide(s) to form the metal oxide(s).

18. The method according to claim 16 or 17, wherein the catalyst is amorphous, or from 0.1 to 50% by weight of the catalyst is in the form of one or more crystalline compounds of chromium and/or one or more crystalline compounds of zinc.

19. (canceled)

20. (canceled)

21. (canceled)

22. (canceled)

23. (canceled)

24. (canceled)

25. (canceled)

26. A method comprising the fluorination and/or hydrofluorination of a halogenated hydrocarbon in the presence of the catalyst prepared according to claim 12.

27. The method according to claim 26, wherein the halogenated hydrocarbon is a (hydro)haloalkene.

28. The method according to claim 27, wherein the (hydro)haioalkene is a C₂₋₇ (hydro)haloalkene.

29. The method according to claim 28, wherein the C₂₋₇ (hydro)haloalkene is a C₂₋₇ (hydro)chlorofluoroalkene.

30. The method according to claim 29, wherein the C₂₋₇ (hydro)chlorofluoroalkene is 2-chloro-3,3,3-trifiuoropropene (1233xf).

31. A process for preparing a fluorinated hydrocarbon, the process comprising reacting an optionally halogenated hydrocarbon with hydrogen fluoride in the presence of the catalyst produced in accordance with the method of claim 12, wherein the process is carried out in vapour phase.
